(19) United States
(12) Patent Application Publication
(10) Pub. No.: US 2022/0133527 A1
(43) Pub. Date: May 5, 2022

(54) Lower Body Protective Device

(71) Applicant: Yvonne Brown, Jamaica, NY (US)

(72) Inventor: Yvonne Brown, Jamaica, NY (US)

(21) Appl. No.: 17/199,435

(22) Filed: Mar. 11, 2021

Related U.S. Application Data
(60) Provisional application No. 63/109,274, filed on Nov. 3, 2020.

(51) Int. Cl. A61F 6/04; A41B 9/00; A41B 9/12; A41B 9/02

(52) U.S. Cl. CPC A61F 6/04; A41B 9/007; A61F 2006/047; A41B 9/026; A61F 2006/041; A41B 9/12

(57) ABSTRACT

Disclosed is a protective garment and a protective device that are intended to limit skin-to-skin exposure during a sexual intercourse between partners. The protective garment reveals only the required areas of the sexual organs, concealing all other surfaces surrounding such organ. The aperture of the protective garment is then removable closed using a flap. A protective device is further disclosed to protect skin contact with protruding elements of a male reproductive organ. The protective device may be used in addition to a standard condom or may be a urinary or an additional feature to a condom.

### CLAIM OF PRIORITY

This application claims benefit to the Provisional Utility Application No. 63/109,274 to the aforenamed inventor, that was filed on Nov. 3, 2020, the entire contents of which are incorporated herein by reference.

### FIELD OF THE INVENTION

The present invention relates to a garment and accessories to be used in prevention and countermeasures to sexually transmitted diseases and urinary incontinence.

### BACKGROUND OF THE INVENTION

Presently the use of a condom during a sexual act is the most recommended means of preventing the spread of an STD. Condoms are also the most basic method of preventing an unwanted pregnancy. The function of a condom is to stop the spread of STDs and sexually transmitted infections STIs or prevent pregnancy by creating an effective barrier to semen.

However, skin cover by a condom is limited to approximately 80% of the shaft of a penis during an erection. Most condoms do not reach the base of the shaft and certainly do not cover the scrotum or the pubic area of the wearer, or any of the exposed skin areas of the sexual partner.

Unfortunately, some STDs, such as, Herpes or Syphilis present sores or lesions in the pelvic area of the infected person, or in a vaginal or anal area of sexual partners, as well as in other areas of the lower body. These areas will still be exposed and are likely to come into contact with exposed skin of a sextual partner or with the partner's sexual organ even when a condom is used. It is therefore still desirable to have an appropriate protection accessory even when using a condom.

Furthermore, most undergarments are not suitable to be worn during a sexual act. While some specialty garments exist, they are not typically sold through regular channels and will therefore be difficult to obtain. It is therefore desirable to have a conventional garment that also doubles as a barrier to skin contact during intercourse without intruding or obstructing a sexual act from occurring.

At the conclusion of a sexual act, individuals may wish to get dressed. However, at that time they may still be emitting body fluids, which then penetrate their garments causing embarrassing and unseemly stains. The same applies in general to those you suffer from sporadic incontinence. A flap having absorbent qualities will prevent these instances and allow the wearer escape embarrassing and awkward situations.

Furthermore, the problem of incontinence plagues many individuals, both young and old. Incontinence is sometimes brought about by a muscular condition, an illness, a mental state or psychological disorder of an afflicted party. Incontinence is an embarrassing condition that requires one to put on specially made garments or barriers to limit emission of odors or fluids. Unfortunately, the devices designated for such use are unfit for use as blockers of STDs as such devices are generally removed prior to intercourse.

### SUMMARY OF THE INVENTION

The disclosed invention is an undergarment comprising a top opening and two bottom openings. The top opening is configured to be worn over a lower body area of a torso with each of the bottom openings admitting one leg of the wearer. The disclosed garment further comprises an aperture, the aperture occurring in a sidewall of the undergarment; the aperture configured to be located opposite a sexual organ of the wearer. A removable flap that is affixed on at least one end, where the removable flap contains at least one fastener for removable coupling with the sidewall of the undergarment. The removable flap is configured to prevent leakage of semen before or after a sexual act as well as to capture other body fluids being emitted through the urethra or anus of an incontinent individual.

The disclosed aperture in the sidewall of the undergarment may be configured to be opposite the penis of a wearer, that is substantially in the front of the garment, or between the legs or in the rear of the wearer, such that the opening is opposite vaginal area or anus of the wearer. The flap is removably or permanently attached along one of its ends to an area of the sidewall, such that the point of attachment acts as a pivot permitting the flap to be peeled off its fastener to reveal the aperture or to be reattached to the fastener, covering the aperture. It is preferred that the flap further comprise an absorbent disposable or reusable pad that is urinary with or separate from such flap. This absorbent pad serves the role of capturing all biofluids being emitted by the wearer.

The present application further discloses a protective device for penis and areas immediately adjacent thereto that can be used alone or in combination with the disclosed undergarment. The protective device is comprised of a conical wall having a first opening and a second opening. The first opening being larger than the second opening. The conical wall is slanted in the direction of the second opening. There are at least two flexible flaps attached near or along a portion of a ring forming the first opening. These two flaps being in a parallel and spaced apart relation with each other. The first opening is configured to be inserted over a penis and scrotum of a wearer, such that said second opening will be admitting a penis of a wearer inserted therethrough.

The disclosed protective device may further comprise a top flap. The top flap is attached axially along the conical wall near the top portion of the ring forming the first opening. The top flexible flap being in a contiguous and overlapping orientation with each of the side flaps. It is appreciated by one skilled in the art that all flaps as well as the conical wall may be made of opaque, transparent or semitransparent materials and be made of flexible materials. A person of such knowledge and skill may be further appreciate that the flaps disclosed may instead be a single flap attaching axially near the rim of the ring forming the first opening. Appreciably, one should bear in mind that the disclosed protective device can function in a dual role of a shield to semen and protection of surrounding skin, as well as to control incontinence symptoms of a wearer.

When used in combination with the disclosed undergarment, the protective device may be permanently affixed or worn as a separate accessory to such garment.

### BRIEF DESCRIPTION OF THE DRAWINGS

FIG. 1 is a front view of a male undergarment, or a back view of a unisex undergarment.

FIG. 2 is the same view as in FIG. 1, showing the flap in a closed state.

FIG. 3 is an alternative embodiment of the undergarment, featuring longer pant legs and a round aperture.

FIG. 3A demonstrating the removable flap that can be used to capture all bodily emissions, such as semen, urine, fecal or blood.

FIG. 4 is the same view as in FIG. 3, with the flap in a closed state.

FIG. 5 demonstrates a combination between the protective undergarment and a protective device.

FIG. 6 demonstrates a female undergarment, with an aperture shown between leg openings.

FIG. 7 is a diagram of the sexual protective device.

FIG. 8 is another diagram of the protective device featuring an alternative embodiment.

FIG. 9 is another embodiment of the figure shown in 7 and 8.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

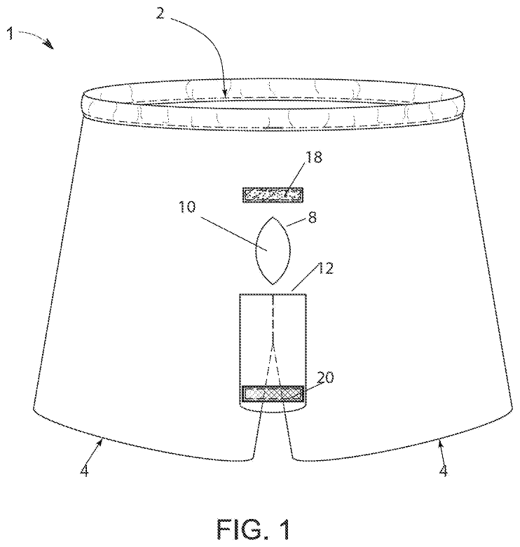

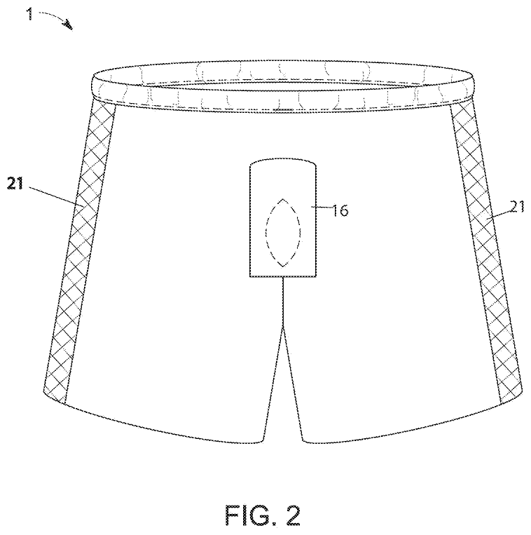

Turning now descriptively to the drawings, in which similar reference characters denote similar elements throughout the several views. FIGS. 1 and 2 demonstrate a male or unisex undergarment 1. The undergarment 1 may double as shorts or as a bathing garment.

Shown in FIGS. 1 and 2 is the top opening 2, the bottom openings 4 and the sidewall 6. The top opening 2 forms the elastic band of the undergarment that fits over the pelvic and posterior portions of a wearer's body. The bottom openings 4 are for wearer's two feet. The aperture 10 is configured to appear opposite a sexual organ of a wearer. In the embodiment shown this aperture 10 may serve as an opening for a penis or an anus of the wearer.

The flap 16 is affixed at least one point 12 near the aperture. It is then removably affixes using the fastener elements 18 and 20 to selectively cover or uncover the aperture 10. The fastener elements 18 and 20 may be a hook and loop fastener, a snap fastener, a hook with a catch combination, or button with buttonholes or two portions of a magnetic fastener. In FIG. 2 the undergarment 1 is shown having a lace design 21 on the side of each leg 4. It should be noted that, as to not defeat the purpose of shielding the skin with such lace design, as well as other lace designs appearing in various figures of this disclosure, the transparency is achieved using impermeable plastic, rubber or polyethylene materials covered with lace, or having an embedded lace design.

The edge 8 of the aperture 10 is elliptical, with sharp points of the ellipse coming to a point at top and bottom. The edge 8 may be rounder. The edge 8 may further contain an elastic or crimpled band around the aperture 10.

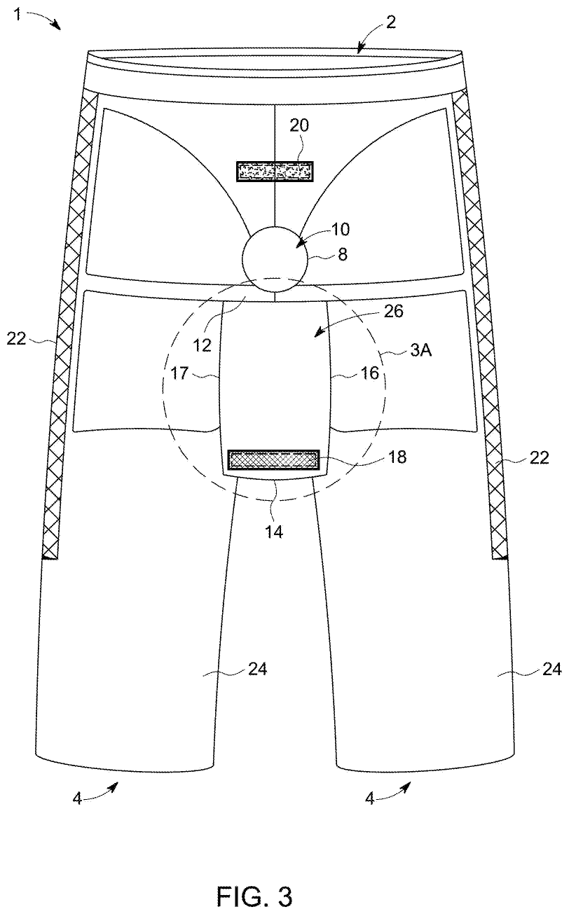

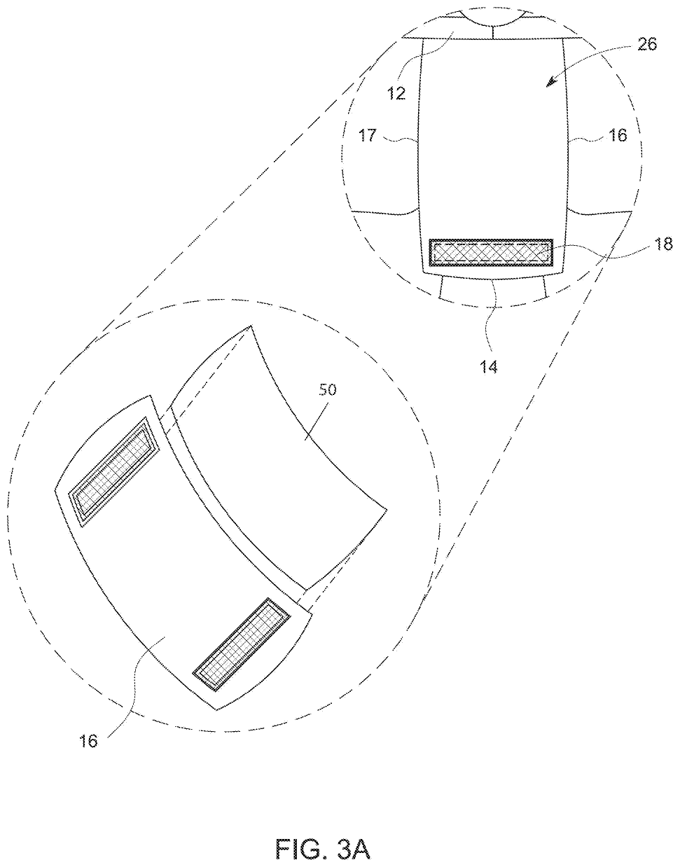

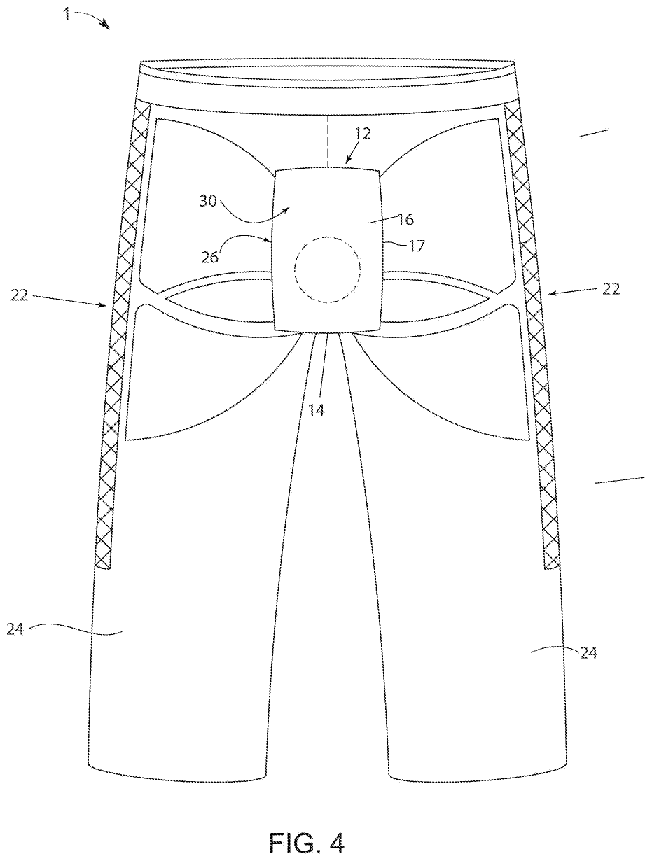

Shown in FIGS. 3 and 4 is an alternative embodiment of the disclosed protective garment. The pant legs 24 are elongated to offer additional protection against skin contact along the surfaces of the wear's legs. As in the FIG. 2, the embodiment shown in FIGS. 3 and 4 demonstrates a transparent pattern 22. This feature may be appealing to a wearer as a perceived libido increasing feature of the invention.

The flap 16 may preferably contain an interior surface 26 and an exterior surface 30. The interior surface may be made of softer, more absorbent materials, while the exterior service 30 is preferred to be made of liquid blocking materials. In this fashion, the flap 16 doubles as a protective device to capture body fluids emitted involuntarily by the wearer. The fastener on the flap 16 is shown to be near the distal end 14, which pivots about the point of affixation 12. Alternatively, multiple fasteners may be disposed all along the edge 17.

The aperture 10 is shown to be circular. As mentioned above, the actual shape of the aperture 10 is not critical to the invention. Other possible shapes may be more square, trapezoidal, triangular or a heart shape or a as petals of a flower.

FIG. 3A is an expanded diagram of the flap 16. The flap is pivotedly attached at point 12. The attachment at point 12 may be temporary or permanent. A removable pad 50, which may be an integrated lining, is placed on the surface of the flap 16 that will come into contact with the wearer of the device. The pad 50 is designed to capture the flow of body fluids of an incontinent or aroused individual and to also limit the emission of odor. It is therefore preferred that the pad 50 contain an absorbent surface facing the skin of the wearer and an impermeable surface on the opposite surface of the pad 50 to seal the fluids within. The pad 50 may be removable for cleaning and disinfection or disposable.

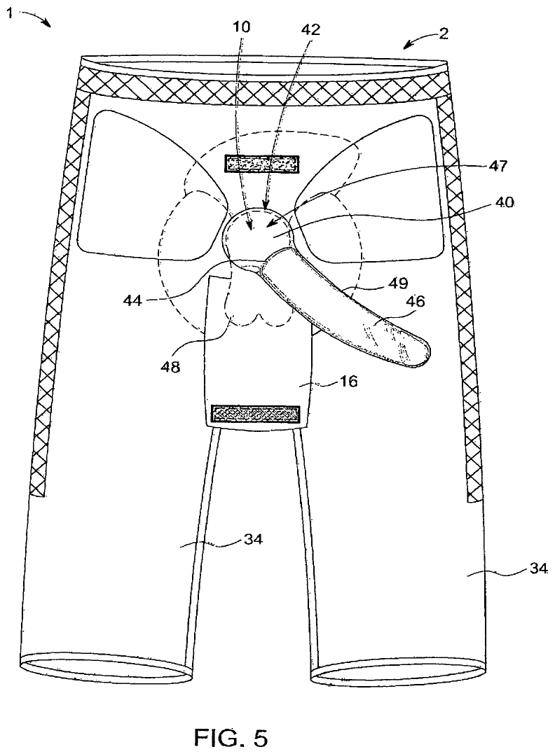

FIG. 5 demonstrates a combination of the protective undergarment 1 with the protective device 40. Shown in the FIG. 5 is the conical wall 47, the first opening 42, and the second opening 44. The sexual organs 46 and 48 of the wearer may pass through the aperture 10, with the conical wall 47 covering the exposed skin at the base of the shaft 46 and the scrotum 48. The rest of the sexual organ 46 is covered by a condom. Alternatively, the condom 49 may be a singular unit with the conical wall 47.

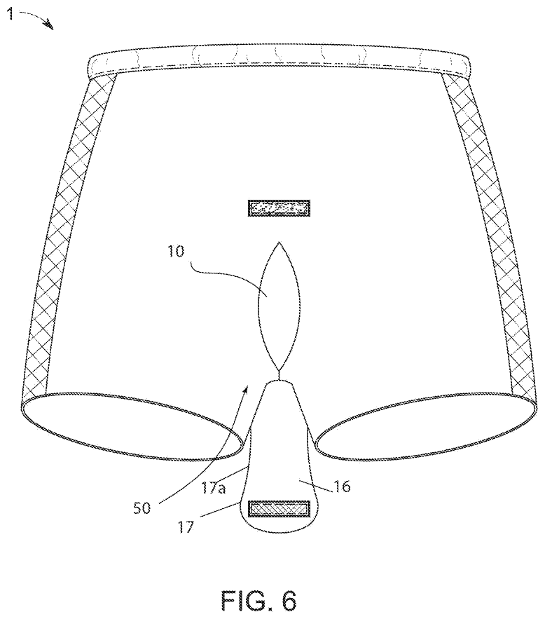

A female undergarment is shown in FIG. 6, with the aperture 10 appearing substantially in the area 50 between the legs of the wearer. The flap 16 is affixed in the back of the aperture 10, which in this case appears to be substantially below the buttocks. The edge 17 of the flap 16 is contoured, especially in the area 17a to avoid interfering with leg movements of the wearer.

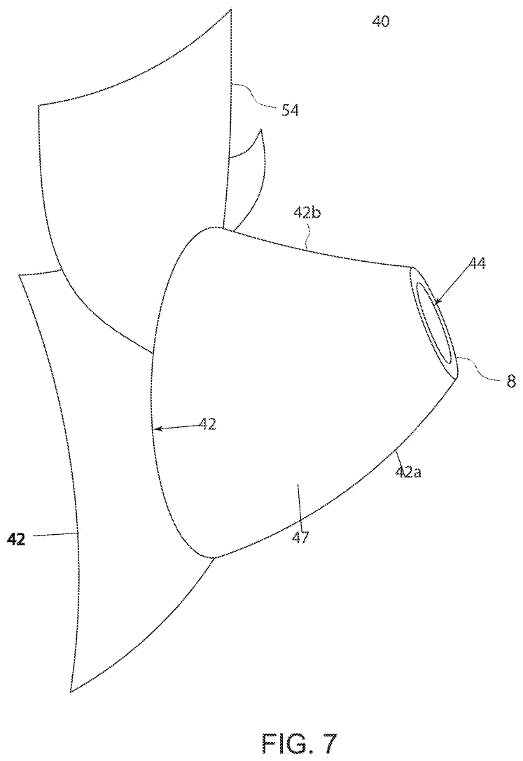

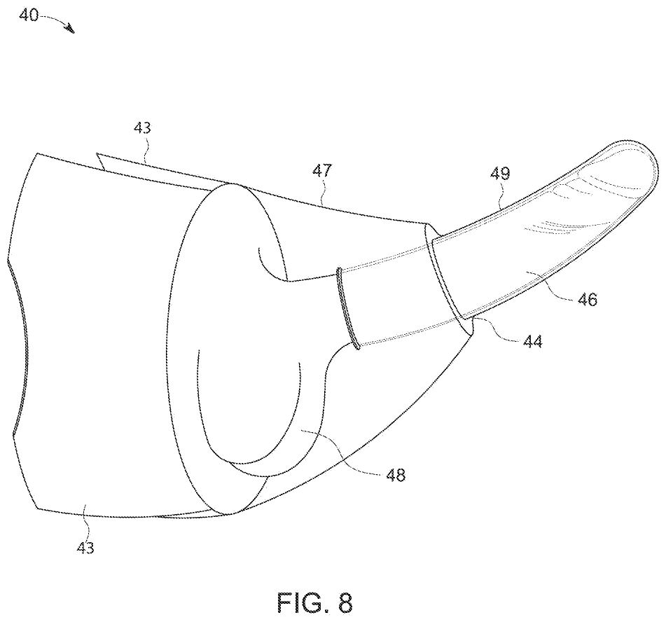

FIG. 7 describes the protective device 40. Shown is the conical sidewall 47, which slopes from a larger opening 42 toward the smaller opening 44. The larger opening 42 is intended to be inserted over the penis 46 and cover capture the scrotum 48 of a wearer (see FIG. 8). The second opening 44 may be oriented slightly upwardly where the top wall 42b is slightly concave and 42a is slightly convex, to accommodate the curvature of an erect organ. Alternatively, the preferably flexible sidewall 47 may form a perfect cone and flex to any side as necessary.

The flexible flaps 43 are attached near the ring forming the opening 42 and are preferably made of pliable and stretchable material, that is substantially impervious, such as a resinous, rubber or polymeric surface. Additionally, the top flap 54 is mounted near the ring forming the first opening 42. The top flap 54 being in an overlapping orientation with each of the parallel flaps 43.

FIG. 8 is a contextual figure of the protective device 40. It should be noted that the condom 49 may be urinary with the sidewall 47. Thus way, the penis 46, passing through the second opening 44 is retained within the condom 49 without the need of deploying the condom 49 and the protective device 40 as separate steps.

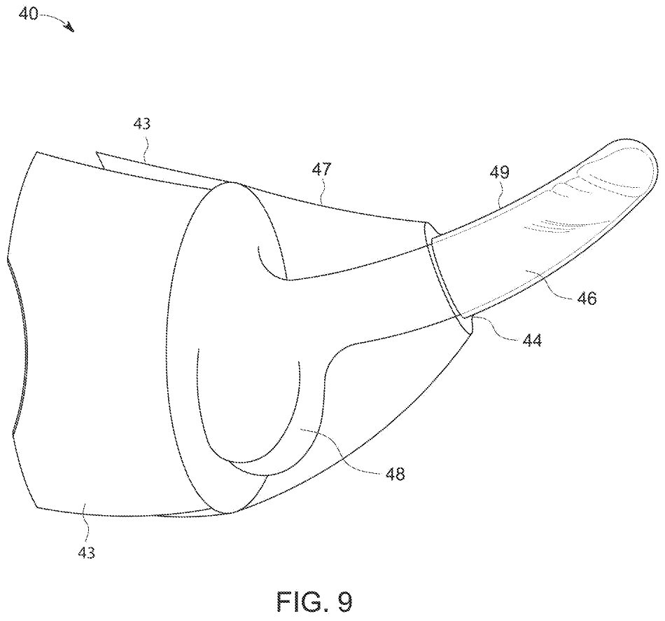

FIG. 9 is a representation of an alternative embodiment of the protective device shown in FIGS. 7 and 8. In FIG. 9 the conical wall 47 is unitary with the condom 49 and are fused at the base 44. This embodiment is most suitable to accomplish roles both as a sexual protective device and an incontinence control device, since it can be quickly deployed and removed, and is more impermeably since the base 44 is sealed.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed:

1. An undergarment comprising a top opening and two bottom openings, wherein the top opening is configured to be worn over a lower body area of a torso with each of said bottom openings admitting one leg of a wearer; an aperture, said aperture occurring in a sidewall of said undergarment, said aperture configured to be located opposite a sexual organ of a wearer; and a removable flap, said flap affixed on at least one side, wherein said removable flap having at least one fastener for removable coupling with said sidewall; and wherein said flap is configured to capture all bodily liquids.

2. The undergarment of claim 1, wherein said aperture is configured to be presented opposite a penis of a wearer, wherein said removable flap is permanently attached in an area of said sidewall behind a scrotum of a wearer; and wherein said at least one fastener being position above said aperture.

3. The undergarment of claim 1, wherein said aperture is configured to be presented opposite a vaginal area of a wearer, wherein said removable flap is permanently attached in an area of said sidewall behind at bottom of a buttocks of a wearer, and wherein said at least one fastener being position above said aperture.

4. The undergarment of claim 1, wherein said aperture is configured to be presented opposite an anus of a wearer, wherein said removable flap is permanently attached in an area of said sidewall behind at bottom of a buttocks of a wearer; and wherein said at least one fastener being position above said aperture.

5. The undergarment of claim 1, wherein said flap further comprising an absorbent surface, said absorbent surface preventing an emission of liquid by a wearer's organ to penetrate said sidewall.

6. The undergarment of claim 1, wherein the sidewall contains transparent pattern along a thigh area of the wearer.

7. The undergarment of claim 5, wherein the sidewall contains transparent pattern along a thigh area of the wearer.

8. The undergarment of claim 5, wherein said liquids a comprised from group of semen, urine, feces or blood, or any combination thereof.

9. A protective device comprising a conical wall having a first opening and a second opening; wherein said first opening being larger than said second opening; and wherein said conical wall beveled in the direction of said second opening; at least two flexible flaps, said at least two flaps attached along a portion of a ring forming said first opening, said at least two flaps being in a parallel and spaced apart orientation with each other; and wherein said first opening is configured to be inserted over a penis and scrotum of a wearer, such that said second opening will be admitting a penis of a wearer inserted therethrough.

10. The protective device of claim 9, further comprising a top flap, said top flexible flap attaching near the top portion of the ring surrounding said first opening; and said top flexible flap being in a contiguous and overlapping orientation with each of said two flaps.

11. An undergarment and protective device combination comprising, an undergarment comprising a top opening and two bottom openings, wherein the top opening is configured to be worn over a lower body area of a torso with each of said bottom openings admitting one leg of a wearer; an aperture, said aperture occurring in a sidewall of said undergarment, said aperture configured to be located opposite a sexual organ of a wearer; a removable flap, said flap permanently attached on at least one side, wherein said removable flap having at least one fastener for removable coupling with said sidewall; wherein said aperture permits unobstructed passage of a sexual organ therethrough; a protective device comprising a conical wall having a first opening and a second opening; wherein said first opening being larger than said second opening; and wherein said conical wall beveled in the direction of said second opening; at least two flexible flaps, said at least two flaps attached along a portion of a ring forming said first opening, said at least two flaps being in a parallel and spaced apart orientation with each other; and wherein said first opening is configured to be inserted over a penis and scrotum of a wearer, such that said second opening will be admitting a penis of a wearer inserted therethrough

12. The protective device of claim 11, further comprising a top flap, said top flexible flap attaching near the top portion of the ring surrounding said first opening; and said top flexible flap being in a contiguous and overlapping orientation with each of said two flaps.

13. The protective device of claim 11, wherein said device is configured to function as a protective device against sexually transmitted diseases and as a protection against urinary incontinence.
